(19) United States
(12) Patent Application Publication
(10) Pub. No.: US 2006/0154259 A1
(43) Pub. Date: Jul. 13, 2006

(54) Diamond crystallites for biotechnological applications

(76) Inventors: Huan-Cheng Chang, Taipei City (TW); Chau-Chung Han, Taipei City (TW)

Correspondence Address: FISH & RICHARDSON PC, P.O. BOX 1022, MINNEAPOLIS, MN, 55440-1022, US

(21) Appl. No.: 11/031,601

(22) Filed: Jan. 7, 2005

(51) Int. Cl. C12Q 1/68; G01N 33/53

(52) U.S. Cl. 435/6; 435/7.1

(57) ABSTRACT

A diamond-based composition that contains (1) a diamond crystallite having chemically derivatized surface groups, and (2) a polymer having functional groups, in which a portion of the functional groups bind to the chemically derivatized surface groups non-covalently. Also disclosed are methods of using such composition for analyzing a biological sample by determining the identity of biomolecules bound to the composition.

### BACKGROUND

Immobilization of biomolecules is a key procedure in many biotechnological applications, including biochips and biosensors. See, e.g., Drummond et al. (2003) Nature Biotech. 21:1192-1199; Zhu et al. (2003) Curr. Opin. Chem. Biol. 7:55-63; Wulfkuhle et al. (2003) Nature Rev. Cancer 3:267-275; Tirumalai et al. (2003) Mol. Cell. Proteomics 2:1096-1103. Different approaches have been developed to anchor biomolecules on solid supports.

For example, micrometer-sized agarose beads made for affinity chromatography columns have been used to capture proteins of interest from crude sample solutions. The agarose beads can then be recovered and analyzed with matrix-assisted laser desorption/ionization (MALDI) time-of-flight (TOF) mass spectrometry (MS). See Hutchens et al. (1993) Rapid Commun. Mass Spectrom. 7:576-580. Direct analysis of the surface-bound proteins is, however, often accompanied with reduced mass resolution and accuracy ascribed to the interference from the agarose beads in ion formation and extraction.

As another example, diamond has been employed as a material for immobilizing biomolecules for bioanalytical applications, given its optical transparency, chemical stability and biological compatibility. See Tang et al. (1995) Biomater. 16:483-488; Hauert et al. (2003) 12:583. Chemically derivatized surface groups can be introduced to the surface of a diamond for coupling to biomolecules. See Miller (1999) Surf. Sci. 439:21-33; Miller et al (1996) Langmuir 12:5809-5817; Yang et al. (2002) Nat. Mater 1:253-257; Strother et al. (2000) J. Am. Chem. Soc. 122:1205-1209; Ushizawa et al. (2002) Chem. Phy. Lett. 351:105-108. Current immobilization methods, however, are often too time-consuming and laborious for general use.

There is a need to develop a diamond-based composition which can be conveniently used for immobilization and, optionally, subsequent analysis of biomolecules.

### SUMMARY

In one aspect, the present invention features a diamond-based composition which includes (1) a diamond crystallite having a surface that contains chemically derivatized surface groups, and (2) a polymer having multiple functional groups. The chemically derivatized surface groups bind to a portion of the functional groups non-covalently, thereby allowing the polymer to cover the surface of the diamond crystallite.

The chemically derivatized surface groups can be amino, carboxyl, carbonyl, phosphate, or hydroxyl groups. The functional groups can be any chemical entities which interact with the chemically derivatized surface groups to form non-covalent bonding. For example, when the functional groups are ionizable groups, they form ionic bonding with the chemically derivatized surface groups that are also ionizable groups but of the opposite charge. As another example, negatively charged surface phosphate group can interact with alkaloids through an intervening metal cation to form a “salt-bridge.” As still another example, hydrogen bonding can be formed between surface hydroxyl groups and protonated amino groups. As yet another example, hydrophobic bonding can also be formed among carbonyl derivatives having a long hydrocarbon chain, e.g., a C18 chain.

Not only can the functional groups bind to the chemically derivatized surface groups, they can also bind to a crosslinking agent, biomolecule, organelle, or cell. Of note, the crosslinking agent possesses at least two reactive groups for respectively binding to one of the chemically derivatized surface groups and binding to a biomolecule, organelle, or cell.

Below are four useful diamond-based compositions of this invention:

In Composition (1), the functional groups that are not bound to the chemically derivatized surface groups are unoccupied. One can use this composition to bind a biomolecule, organelle, or cell via interaction with the unoccupied functional groups either covalently or non-covalently. As an example of covalent interaction, a disulfide bond can be formed between biomolecules and functional groups.

Composition (2) differs from Composition (1) only in that at least one of the functional groups is covalently bound to a reactive group of a crosslinking agent, which has at least one additional reactive group that is unoccupied. This composition can be used to bind a biomolecule covalently through the unoccupied reactive group.

Composition (3) differs from Composition (1) only in that a biomolecule, organelle, or cell is bound covalently or non-covalently to the unoccupied functional groups. When this composition contains a biomolecule of known molecular weight, it can be used as a standard for comparison with another composition that contains a biomolecule to be identified. This composition can also be used to bind a second biomolecule, organelle, or cell through the first biomolecule, organelle, or cell. Such a composition is also within the scope of this invention. For example, yeast cytochrome c (YCC), which possesses a free surface sulfhydryl group, can be used as a first biomolecule to bind a second biomolecule having a free cysteine residue by disulfide bonding.

Composition (4) differs from Composition (2) in that a biomolecule, organelle, or cell is covalently bound to the unoccupied reactive group. Similar to Composition (3), when this composition contains a biomolecule of known molecular weight, it can be used as a standard for comparison with another composition that contains a biomolecule to be identified. This composition can also be used to bind a second biomolecule, organelle, or cell through the first biomolecule, organelle, or cell. Such a composition is also within the scope of this invention.

In another aspect, this invention relates to methods for analyzing a biological sample such as serum. For example, one can mix the sample and Composition (1) to allow a molecule of interest in the sample to bind non-covalently to Composition (1), and identify the molecule by, e.g., mass spectrometry. As another example, one can mix the sample and Composition (2) to allow a molecule of interest in the sample to bind covalently to Composition (2), and then identify the molecule.

Other features, objects, and advantages of the invention will be apparent from the description and from the claims.

### DETAILED DESCRIPTION

This invention relates to a diamond-based composition which includes (1) a diamond crystallite having chemically derivatized surface groups, and (2) a polymer having functional groups. The diamond crystallite is coated with the polymer through non-covalent interaction between the chemically derivatized surface groups and the functional groups.

The term “diamond crystallite” refers to a diamond powder whose size is 1 nm to 100 μm in diameter (e.g., 5 nm to 20 μm). The size of the diamond crystallites is selected based on the applications and the analysis techniques employed. For example, 100 to 500 nm diamond crystallites are most useful for separating diamond-bound biomolecules by centrifugation. As another example, 1 to 100 μm ones are required for column chromatography. The term “diameter” is defined as the distance between the two longest points on a diamond crystallite. The size of a diamond crystallite can also be described by aspect ratio, which is defined as the ratio of the longest to the shortest linear dimensions. For example, the diamond crystallites in Compositions (1) to (4) preferably have an aspect ratio of 1 to 2. The size of diamond crystallites can be measured either by mechanical sieving (for micrometer-sized powders) or by various electron microscopy, e.g. scanning and transmission electron microscopies (for nanometer-sized powders).

To prepare a diamond crystallite of this invention, the diamond surface is first modified to generate chemically derivatized surface groups. The term “chemically derivatized surface group” refers to amino, carboxyl, carbonyl, hydroxyl, amide, nitrile, nitro, diazonium, sulfide, sulfoxide, sulfone, sulfhydryl, epoxyl, phosphoryl, oxycarbonyl, sulfate, phosphate, imide, imidoester, pyridinyl, purinyl, pyrimidinyl, and guanidinyl groups. They can be introduced to the diamond surface using classical organic synthesis procedures with minor modifications. For example, carboxyl groups can be introduced to the diamond surface by oxidative acid treatment as described in Example (1) below. Other chemically derivatized surface groups can be derived from the starting carboxyl group. For example, amide groups can be generated by reacting the carboxylated diamond crystallites in concentrated NH₃ solution at room temperature for one day. Amino groups can be introduced to diamond surface by treating carboxylated diamond crystallites in thionyl chloride at 50° C. for one day, followed by ethylenediamine under reflux for one day. Carbonyl groups are generated by first converting carboxyl groups into acyl chloride or bromide groups, followed by an S_{N}2 or S_{N}1 alkyating reaction. For those chemically derivatized surface groups that are ionizable, they can form ionic bonding with the functional groups that also have ionizable groups but are of the opposite charge. The term “ionizable group” refers to the chemical group that is capable of forming ions in solution at a given pH. Examples of ionizable groups include amino, carboxyl, hydroxyl, amide, sulfide, sulfhydryl, imide, pyridinyl, purinyl, pyrimidinyl, and guanidinyl groups.

The term “polymer” covers macromolecules such as polypeptide, polysaccharide, nucleic acid, industrial polymers (e.g., polystyrene, polyesters, polyethyleneglycols, and polyvinyl halides), and their derivatives. These polymers must contain a number of functional groups so that they can interact with the chemically derivatized surface groups. For example, a poly-L-lysine with molecular weight of 3,000 to 30,000 (e.g., 10,000) can be employed to coat a carboxylated diamond crystallite. As another example, a poly-L-arginine can also be used. In these two examples, the key functional groups are both amino groups.

Note that in Composition (1), described in the Summary section, the functional groups that are not bound to the chemically derivatized surface groups are unoccupied. In Composition (2), also described in the Summary section, a crosslinking agent having two or more reactive groups is attached to Composition (1) via covalent bonding between the reactive group and one of the unoccupied functional groups. The term “crosslinking agent” refers to heterofunctional chemical crosslinkers, each having two or more reactive groups. One of the reactive groups binds covalently to the functional group of the polymer, whereas another is unoccupied and thus available for further desired manipulation. Examples of such crosslinking agents include sulfosuccinimidyl 4-(N-maleimidomethyl)-cyclohexane-1-carboxylate (SSMCC), γ-maleimidobutyric acid N-hydroxysuccinimide ester (GMBS), N-[α-maleimidocaproyloxy]succinimide ester), N-[α-maleimidocaproyloxy]sulfosuccinimide ester, ethylene glycolbis(succinimidylsuccinate), and 3-[(2-aminoethyl)dithio]propionic acid, and N-(α-maleimidoacetoxy)succinimide ester. The chemical properties of these crosslinking agents have been well characterized. For example, SSMCC is a heterobifunctional crosslinker. One end of SSMCC reacts with the amino group of a polymer-coated surface, whereas the other end reacts specifically with a sulfhydryl group of a cysteine-containing protein. As another example, GMBS functions as a crosslinking agent between sulfhydryl groups of a polymer-coated surface and lysine amino groups of a protein.

Composition (3), described in the Summary section, is a diamond-based composition containing a diamond crystallite coated with a polymer bound to a biomolecule, organelle or cell. The term “biomolecule” encompasses individual molecules and molecular complexes. Individual molecules include protein, nucleic acid, polysaccharide, lipid, and their derivatives. Examples of individual molecules, therefore, include hydrophobic hydrocarbon chain; hydrophilic chains containing one or more carboxyl, sulfhydryl, hydroxyl, sulfoxide, sulfonyl, amino, pyridinyl, ammonium, carbonyl, sulfate, and phosphate groups; chelating agents; antigens such as peptidoglycan and lipoglycan; antibodies; DNA; lipoprotein, cholesterol, and sphingolipid; carbohydrates and their derivatives such as glycoprotein; metabolites such as ATP and NAD; hormones such as lipid derivatives; amino acid derivatives; and polypeptides. Almost any proteins or polypeptides with MW>2,000 can bind non-specifically to polymer-coated diamond crystallites. For small proteins (e.g., gramicidin-S and bradykinin) or polypeptides, the binding can also occur as long as they carry a net charge which is opposite to that of a polymer. As for nucleic acid, the binding occurs non-specifically and almost all oligodeoxynucleotides can bind to polymer-coated diamond. Examples of oligodeoxynucleotide include dpT₁₆, dpC₁₆, dpA₁₆, dpG₉g, dATCGGCTAATCGGCTA (a 16-mer), and lambda gt11 (forward, dGGTGGCGACGACTCCTGGAGCCCG). For example, positively charged amino groups on poly-L-lysine can form ionic bonds with negatively charged phosphate groups on dpA16 at neutral pH. Molecular complexes include protein-protein assemblages, protein-polynucleotide assemblages, and liposomes. A molecular complex can be an antibody, virus capsid, or liposome. The term “organelle” refers to sub-cellular structure in an unicellular or multi-cellular organism. Examples include nucleolus, mitochondrion, ribosome, lysosome, golgi body, endosome, and endoplasmic reticulum. The term “cell” refers to the basic structural and functional unit of a unicellular or multi-cellular organism. Examples include bacterial cells, somatic cells, adult stem cells, and embryonic stem cells.

DNA immobilized on diamond surface can be digested enzymatically into smaller fragments (e.g., cleavage by phosphodiesterases in both 3′ to 5′ and 5′ to 3′ directions), followed by sequence analysis with MALDI-TOF-MS. Liposome has been widely explored as a drug carrier. One can preserve the stability (i.e., prolong the half-life) of a drug-carrying liposome by coupling it to a diamond crystallite. One can also use diamond crystallites to facilitate isolation and analysis of viral particles by immobilizing viruses on diamond surfaces.

Composition (4), also described in the Summary section, is a diamond crystallite coated with a polymer that is bound to a crosslinking agent which further binds to a biomolecule, organelle, or cell. Organelle and cell can be coupled to a diamond surface through covalent bonding with a crosslinking agent in the manner described in Shriver-Lake et al. See Shriver-Lake et al (2002) Analytica Chimica Acta: 470:71-78. To both Compositions (3) and (4), the biomolecule, organelle, or cell can further bind to a second biomolecule, organelle, or cell. The second biomolecule, organelle, or cell can be coupled to a diamond surface by covalently attaching an antibody (i.e., the first biomolecule) to a crosslinking agent that is bound to a polymer-coated diamond crystallite. The antibody, which recognizes its corresponding antigen such as a polypeptide sequence or a glycol- or lipo-derivative thereof, can specifically bind to its antigen-bearing target such as a molecule, molecular complex, organelle, viral capsid, liposome, or cell.

This invention further features a method for analyzing a biological sample by mixing the sample and Composition (1) to allow a molecule of interest in the sample to bind non-covalently to Composition (1), and determining the identity of the molecule. The term “biological sample” refers to any specimen originated from a living organism. Examples include extracts of cellular contents, tissue biopsy sections, breast milk, gastric fluid, bronchial fluid, cerebrospinal fluid, ascetic fluid, utero-vaginal discharge, urine, feces, semen, menstrual blood, saliva, sputum, and serum. The identity of the molecule bound to Composition (1) can be determined by standard analytical methods including, but not limited to, MALDI-TOF-MS, sodium dodecyl sulfate-polyacrylamide gel electrophoresis (SDS-PAGE), and enzyme linked immuno-sorbent assay (ELISA).

A second method featured in this invention for analyzing a biological sample is by mixing a sample and Composition (2) to allow a molecule of interest in the sample to bind covalently to Composition (2), and identifying the molecule. Similarly, the identity of the molecules bound to the composition can be determined by standard analytical methods including, but not limited to, MALDI-TOF-MS, SDS-PAGE, and ELISA.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLE 1

Diamond crystallites, 5 to 100 nm in diameter, were functionalized by acid treatment following the procedures described in Ushizawa et al. (2002) Chem. Phy. Lett. 351:105-108. Specifically, the diamond crystallites were first heated in a 9:1 (v/v) mixture of concentrated H₂SO₄ and HNO₃ at 75° C. for 3 days, subsequently in 0.1 M NaOH aqueous solution at 90° C. for 2 hours, and finally in 0.1 M HCl aqueous solution at 90° C. for 2 hours. The resulting carboxylated diamond crystallites were extensively rinsed with de-ionized water and separated by centrifugation with a Kubota 3700 centrifuge at 12,000 rpm. Two stock suspensions, each containing 1 mg and 0.1 mg of diamond crystallites per mL, were prepared with de-ionized water. 0.07 g of the carboxylated diamond crystallites were mixed with 0.03 g of poly-L-lysine in boric acid (10 mL, pH adjusted by NaOH aqueous solution to 8.5) for 30 minutes to obtain diamond crystallites coated with poly-L-lysine which contains amino groups. The poly-L-lysine-coated diamond crystallites thus obtained were then thoroughly washed with de-ionized water.

### EXAMPLE 2

0.07 g of the poly-L-lysine-coated diamond crystallites prepared from Example 1 were mixed with 2.2 mg SSMCC, a heterobifunctional crosslinking agent, in 10 mL phosphate buffer saline at pH 8.5 for one hour to obtain poly-L-lysine/SSMCC-coated diamond crystallites. After separation of excess SSMCC by centrifugation, the sedimentary diamond crystallites were thoroughly washed with de-ionized water.

### EXAMPLE 3

The carboxylated diamond crystallites prepared from Example 1 were used for analyzing human blood serum. Blood serum samples were obtained from healthy males, clotted, and subsequently separated by centrifugation. The serum thus isolated was divided into 50-μl portions and immediately stored in a −20° C. refrigerator until use. Three independent mass analyses of blood serum were conducted for:

(1) Conventional method. 1 μL of blood serum was mixed with 50 μL of 4-hydroxy-α-cyanocinnamic acid (4HCCA) matrix solution, and 2 μL of the serum-matrix mixture was deposited on a stainless steel MALDI-TOF-MS probe and air-dried.

(2) ZipTip method. A ZipTip (C 18 pipette tip, Millipore) containing resin for binding molecules was first activated following the standard protocol of the manufacturer. 50 μL of blood serum was then passed through the ZipTip repeatedly by pipetting the sample solution (10 μL each) in and out 5 times. After rinsing three times with an aqueous solution containing 0.1% trifluoroacetic acid (TFA) and 5% methanol, the molecules attached to the resin were eluted with the 0.001:1:1 (v/v) TFA-acetonitrile-water mixture (10 μL). Half of the elution was mixed with 2 μL of 4HCCA matrix solution and the mixture was deposited on the MALDI-TOF-MS probe.

(3) Diamond crystallite method. 10 μL of blood serum was diluted 100 times with de-ionized water and then mixed with 10 μL of the diamond crystallite suspension (1 mg/mL). After equilibration for 2 minutes, the combined solution was centrifuged for 5 minutes and the supernatant was removed. The precipitate was washed once with de-ionized water (1 mL), collected by centrifugation (3 minutes), and finally mixed with 5 μL of 4HCCA matrix solution. An aliquot (1 μL) of the mixture was deposited on a MALDI-TOF-MS probe for mass spectroscopic measurements.

In the conventional method, each sample was diluted 50-fold directly with 4HCCA matrix solution in order to obtain a mass spectrum with decent signal-to-noise ratios. The spectrum showed three strong signals at m/z 66440, 33220, and 22150 corresponding to human serum albumin; however, it displayed only two distinct features at m/z 2000-10000. In the serum samples purified with the ZipTip method, many new features emerged in the lower m/z region owing to desalting and pre-concentration of the sample. In the serum samples pretreated with diamond crystallites, similar high-quality mass spectra were obtained even though 10-fold less serum was used for data acquisition. Compared to the ZipTip result, the acquired mass spectrum was 5-fold higher in overall peak intensity and was noticeably richer in spectral features over the entire mass range. Furthermore, the albumin peaks were suppressed to a greater extent with the diamond crystallite method than the ZipTip method. Without compromising the high sensitivity as well as the high selectivity, the entire analysis of each sample was finished in as short as 10 minutes.

These results were unexpected, given the significant improvement in sensitivity and accuracy compared to other existing methods.

### EXAMPLE 4

The poly-L-lysine/SSMCC-coated diamond crystallites prepared from Example 2 were used to covalently bind a protein. The crystallites (0.07 g in 10 mL) and 26 μM phosphate-buffered YCC (1.6 mg protein in 5 mL of phosphate-buffered saline at pH 6.5) were mixed for one hour. The resulting protein-diamond mixture underwent several cycles of washing with de-ionized water until the supernatant fraction of the sample appeared clear and transparent after centrifugation, showing negligible absorption at 409 nm.

YCC absorbs strongly at 409 nm (the Soret band) and contains a single free sulfhydryl group (cysteine 102) for covalent bonding with SSMCC, a heterobifunctional crosslinking agent. One end of the crosslinking agent reacted with amino groups of poly-L-lysine coated on diamond crystallites, whereas the other end reacted with a sulfhydryl group of a cysteine-containing protein. In the Fourier transform infrared (FTIR) spectrum of YCC immobilized on the 100 nm poly-L-lysine/SSMCC-coated diamond crystallites, both poly-L-lysine and YCC contributed to the observation of the amide I and II bands in the spectrum. The contribution of the latter, however, was deduced semi-quantitatively by proper normalization of the spectrum with respect to the surface C═O absorption bands at ˜1800 cm⁻¹, followed by subtracting the spectrum of poly-L-lysine in the amide vibration region. Similar analysis applied to YCC on 5 nm poly-L-lysine/SSMCC-coated diamond crystallites indicated that the adsorption density of the covalently immobilized proteins nearly doubled with the aid of SSMCC, compared to the proteins immobilized non-covalently without SSMCC.

A protein stability experiment was also conducted. Two samples, poly-L-lysine/YCC-coated diamond crystallites and poly-L-lysine/SSMCC/YCC-coated diamond crystallites, were deposited separately on Ge(111) wafers and air-dried to generate thin films. The stability of YCC on the thin films was tested using FTIR. The YCC protein on the thin films was so stable that the spectra remained essentially unchanged after 10 cycles of washing. After storage of the sample suspensions at 4° C. for 5 months, the YCC film showed only slight decreases in intensity of both the amide bands, revealing desorption of some non-covalently bound proteins. More remarkably, the poly-L-lysine/SSMCC/YCC film produced a spectrum essentially identical to that of a freshly prepared one, indicating unexpectedly high stability of the immobilized biomolecules.

### EXAMPLE 5

The poly-L-lysine-coated diamond crystallites prepared from Example 1 were used to detect nucleic acid. A matrix solution containing 2,4,6 trihydroxy acetophenone (3HPA), picolinic acid (PA), di-ammoniumhydrogen citrate and TFA was used for detecting DNA with MALDI-TOF-MS. The solution was prepared by dissolving 50 mg 3HPA and 7 mg PA and 10 mg ammonium citrate in 500 μL 50% aqueous acetonitrile with 0.1% TFA. The utility of diamond crystallites for sample volume reduction was demonstrated in the experiment described below.

Oligodeoxynucleotide solutions of different final concentrations (0.1-100 nM) were prepared with de-ionized water. Similar to the procedures described above for protein analysis, an aliquot (500 μL) of the oligodeoxynucleotide solution was mixed with poly-L-lysine-coated diamond crystallites (0.2-10 μL) in a centrifuge tube for 10 minutes. The oligodeoxynucleotide-attached diamond crystallites were precipitated by centrifugation. Upon removal of the supernatant, the matrix solution (1.5-10 μL) was added to resuspend the precipitate. An aliquot (1 μL) of the resuspended solution was deposited on the MALDI-TOF-MS probe and air-dried. The mass spectra for the oligodeoxynucleotide dpA₁₆ with and without diamond crystallite treatment were acquired. In the absence of diamond crystallites, the peaks corresponding to singly and doubly charged negative ions were identified only for the oligodeoxynucleotide at concentration of 40 nM or higher (0.5 μL sample solution mixed with 0.5 μL matrix solution on the MALDI-TOF-MS probe). No signals were identifiable at lower concentrations. With the aid of diamond crystallites to pre-concentrate the oligodeoxynucleotides, signals were identified at the concentration of 4 nM and, remarkably, the detection limit was lowered to 0.4 nM. Similar to the analysis for proteins, the presence of diamond crystallites did not show much adverse effect on performance of the MALDI-TOF-MS.

Other than dpA₁₆, oligodeoxynucleotides composed of C, T, G, and their mixtures were also detected with high sensitivity for singly charged negative ions in the associated m/z region. This finding was unexpected, given the significantly different extent of ion fragmentation among these oligodeoxynucleotides. Even more unexpected was the finding that the mass analysis did not show any sign of interference from protein contaminants, such as ubiquitin, that were 100-fold more abundant than the target molecules.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A diamond-based composition comprising: a diamond crystallite having a surface that contains chemically derivatized surface groups, and a polymer having a plurality of functional groups, wherein the chemically derivatized surface groups are amino, carboxyl, carbonyl, hydroxyl, amide, nitrile, nitro, diazonium, sulfide, sulfoxide, sulfone, sulfhydryl, epoxyl, phosphoryl, oxycarbonyl, sulfate, phosphate, imide, imidoester, pyridinyl, purinyl, pyrimidinyl, or guanidinyl groups; and a portion of the functional groups bind to the chemically derivatized surface groups non-covalently.

2. The diamond-based composition of claim 1, wherein the functional groups that are not bound to the chemically derivatized surface groups are unoccupied.

3. The diamond-based composition of claim 2, wherein both the chemically derivatized surface groups and the functional groups are ionizable groups.

4. The diamond-based composition of claim 1, further comprising a crosslinking agent with two or more reactive groups, wherein one reactive group binds to one of the functional groups covalently and another reactive group is unoccupied.

5. The diamond-based composition of claim 4, wherein the crosslinking agent is sulfosuccinimidyl 4-(N-maleimidomethyl)-cyclohexane-1-carboxylate, γ-maleimidobutyric acid N-hydroxysuccinimide ester, N-[α-maleimidocaproyloxy]succinimide ester), N-[α-maleimidocaproyloxy]sulfosuccinimide ester, ethylene glycolbis(succinimidylsuccinate), 3-[(2-aminoethyl)dithio]propionic acid, and N-(α-maleimidoacetoxy)succinimide ester.

6. The diamond-based composition of claim 4, wherein both the chemically derivatized surface groups and the functional groups are ionizable groups.

7. The diamond-based composition of claim 6, wherein the crosslinking agent is sulfosuccinimidyl 4-(N-maleimidomethyl)-cyclohexane-1-carboxylate, γ-maleimidobutyric acid N-hydroxysuccinimide ester, N-[α-maleimidocaproyloxy]succinimide ester), N-[α-maleimidocaproyloxy]sulfosuccinimide ester, ethylene glycolbis(succinimidylsuccinate), 3-[(2-aminoethyl)dithio]propionic acid, and N-(α-maleimidoacetoxy)succinimide ester.

8. The diamond-based composition of claim 1, further comprising a crosslinking agent with two or more reactive groups, wherein one reactive group binds to one of the functional groups covalently and another reactive group binds to a first biomolecule, organelle, or cell covalently.

9. The diamond-based composition of claim 8, wherein both the chemically derivatized surface groups and the functional groups are ionizable groups.

10. The diamond-based composition of claim 9, wherein the first biomolecule, organelle, or cell is further bound to a second biomolecule, organelle, or cell.

11. The diamond-based composition of claim 10, wherein another reactive group binds to a first biomolecule.

12. The diamond-based composition of claim 11, wherein the crosslinking agent is sulfosuccinimidyl 4-(N-maleimidomethyl)-cyclohexane-1-carboxylate, γ-maleimidobutyric acid N-hydroxysuccinimide ester, N-[α-maleimidocaproyloxy]succinimide ester), N-[α-maleimidocaproyloxy]sulfosuccinimide ester, ethylene glycolbis(succinimidylsuccinate), 3-[(2-aminoethyl)dithio]propionic acid, and N-(α-maleimidoacetoxy)succinimide ester.

13. The diamond-based composition of claim 8, wherein the first biomolecule, organelle, or cell is further bound to a second biomolecule, organelle, or cell.

14. The diamond-based composition of claim 13, wherein another reactive group binds to a first biomolecule.

15. The diamond-based composition of claim 14, wherein the crosslinking agent is sulfosuccinimidyl 4-(N-maleimidomethyl)-cyclohexane-1-carboxylate, γ-maleimidobutyric acid N-hydroxysuccinimide ester, N-[α-maleimidocaproyloxy]succinimide ester), N-[α-maleimidocaproyloxy]sulfosuccinimide ester, ethylene glycolbis(succinimidylsuccinate), 3-[(2-aminoethyl)dithio]propionic acid, and N-(α-maleimidoacetoxy)succinimide ester.

16. The diamond-based composition of claim 1, further comprising a first biomolecule, organelle, or cell bound covalently or non-covalently to the unoccupied functional group.

17. The diamond-based composition of claim 16, wherein both the chemically derivatized surface groups and the functional groups are ionizable groups.

18. The diamond-based composition of claim 17, wherein the first biomolecule, organelle, or cell is further bound to a second biomolecule, organelle, or cell.

19. The diamond-based composition of claim 16, wherein the first biomolecule, organelle, or cell is further bound to a second biomolecule, organelle, or cell.

20. A method for analyzing a biological sample comprising:
providing a diamond-based composition of claim 2, mixing the sample and the composition so that a biomolecule of interest in the sample is bound covalently or non-covalently to the composition through one or more of the unoccupied functional groups, and identifying the biomolecule of interest.

21. A method for analyzing a biological sample comprising: providing a diamond-based composition of claim 4, mixing the sample and the composition so that a biomolecule of interest in the sample is bound covalently to the composition through the unoccupied reactive groups, and identifying the biomolecule of interest.
